(19) United States
(12) Patent Application Publication
(10) Pub. No.: US 2026/0216529 A1
(43) Pub. Date: Jul. 30, 2026

(54) TREATMENT OF CHRONIC PAIN VIA DIRECT PHOTOBIOMODULATION OF A NERVE

(71) Applicant: Case Western Reserve University, Cleveland, OH (US)

(72) Inventors: Andrew BUZZA, Cleveland, OH (US); Michael JENKINS, Cleveland, OH (US); Juanita ANDERS, Cleveland, OH (US); Michael MOFFITT, Cleveland, OH (US)

(21) Appl. No.: 19/150,975

(22) PCT Filed: Jan. 26, 2024
(86) PCT No.: PCT/US2024/013019

§ 371 Date: Jul. 25, 2025

Related U.S. Application Data
(60) Provisional application No. 63/441,325, filed on Jan. 26, 2023.

(51) Int. Cl. A61N 5/06

(52) U.S. Cl. CPC A61N 5/0622; A61N 5/0601; A61N 2005/0612; A61N 2005/0627

(57) ABSTRACT

Direct photobiomodulation (PBM) refers to applying PBM directly to a nerve of interest. Dosages of direct PBM can be configured by a controller/energy source located outside the body and delivered by an emitter implanted within the body proximal to at least one sensory/sensorimotor nerve for chronic pain management. The at least one dose of PBM can be applied for a time to selectively inhibit conduction of signals indicative of pain in at least one small diameter sensory nerve fiber without affecting conduction of at least one larger diameter nerve fiber within the at least one sensory and/or sensorimotor nerve. Then conduction in the small diameter sensory fiber(s) remains blocked for another time (at least a day longer than the time), thereby stopping pain for at least the other time.

### RELATED APPLICATIONS

This application claims priority to U.S. Provisional Application Ser. No. 63/441,325, filed Jan. 26, 2023, entitled “Elucidating photobiomodulation dosing via model-based estimates and direct light application at the nerve”. The entirety of this application is hereby incorporated by reference for all purposes.

### GOVERNMENT FUNDING

This invention was made with government support under T32EB004314 and R01NS121372 awarded by the National Institutes of Health. The government has certain rights in the invention.

### TECHNICAL FIELD

The present disclosure relates to treatment of chronic pain, and more specifically, to systems and methods for treatment of chronic pain via direct application of PBM to a nerve.

### BACKGROUND

Moderate to severe chronic pain is both disruptive and difficult to manage. The most common treatments prescribed for moderate to severe chronic pain are pharmaceutical treatments and electrical nerve block/stimulation treatments. Pharmaceutical treatments often employ opioids (e.g., fentanyl, hydrocodone, morphine, etc.) that can successful treat moderate to severe chronic pain in many instances. However, opioids have many dangerous and unwanted side effects including addiction, hyperalgesia, and overdose. Alternative treatments, like spinal cord stimulation (SCS) and peripheral nerve stimulation (and/or block) (PNS), have been explored as an alternative to pharmaceutical treatments. However, both SCS and PNS are examples of electrical nerve block/stimulation treatments, which are not effective treatments for all types of chronic pain.

### SUMMARY

Direct photobiomodulation (PBM) of a nerve can provide an alternative to pharmaceutical treatments and to unsuccessful electrical nerve block/stimulation treatments. Described herein are systems and methods for treatment of chronic pain via direct application of PBM to a nerve.

In one aspect, the present disclosure includes a method for chronic pain management via direct PBM of a nerve. An external controller can be utilized to configure a dose of PBM having at least one parameter. An emitter, implanted near at least one sensory and/or sensorimotor nerve of a patient, can communicate with the controller for at most a time and apply the dose of PBM directly to the at least one sensory and/or sensorimotor nerve for the time to selectively inhibit conduction in at least one small diameter sensory nerve fiber in the at least one sensory and/or sensorimotor nerve without affecting conduction of at least one larger diameter nerve fiber in the at least one sensory and/or sensorimotor nerve. Conduction of the at least one sensory signal is inhibited for another time that is at least a day longer than the time.

In another aspect, the present disclosure includes a system that can be used for chronic pain management via direct PBM of a nerve. The system can include an implantable emitter configured to be implanted in proximity to at least one sensory and/or sensorimotor nerve of a patient to deliver at least one dose of PBM directly to the at least one sensory and/or sensorimotor nerve at an emitter power to inhibit conduction in at least one small diameter nerve fiber within the at least one sensory and/or sensorimotor nerve without affecting conduction of at least one larger diameter nerve fiber within the at least one sensory and/or sensorimotor nerve. The system can also include external components, located external (outside the body) to the emitter, including an energy source and a controller. The energy source and/or controller can be temporarily coupled to the emitter for at most a time to drive delivery of the at least one dose of PBM from the emitter directly to the at least one sensory and/or sensorimotor nerve. The controller can be coupled to the emitter through the energy source for at most the time and configured to set at least one parameter for the at least one dose of PBM. Application of the at least one dose of PBM for the time selectively inhibits at least a portion of conduction of at least one sensory signal by the at least one small diameter nerve fiber for another time that is at least a day longer than the time.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 shows a system for treatment of chronic pain via direct application of photobiomodulation (PBM) to a nerve;

FIGS. 2 and 3 show examples using the system of FIG. 1 to apply light to block conduction in small sensory nerve fibers;

FIG. 4 shows an example of the external components of FIG. 1;

FIG. 5 shows an example plot of the time course of different PBM blocks; and

FIG. 6-8 show example process flow diagrams of methods for treatment of chronic pain via direct application of PBM to a nerve;

FIG. 9 shows a graphical representation of the power at the emitter and duration of PBM application for four treatment groups tested on the SNI pain model;

FIG. 10 shows plots that illustrate how higher energy PBM reduces hypersensitivities associated with the small-diameter nerve fibers;

FIG. 11 shows plots that illustrate how lower energy PBM reduces hypersensitivities associated with the small-diameter nerve fibers when high power (60 mW) is used;

FIG. 12 shows plots that illustrate how higher energy density PBM does not reduce hypersensitivities when applied to an uninjured nerve (sham SNI surgery); and

FIG. 13 shows plots that illustrate tissue heating through the use of 1833.6 J/cm² (higher power density) is approximately 5° C. with the lower power density (611.2 J/cm²) being 2° C. at the nerve.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

It will be understood that when an element is referred to as being “on,” “attached” to, “connected” to, “coupled” with, “contacting,” etc., another element, it can be directly on, attached to, connected to, coupled with, or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

As used herein, the term “pain” refers to an unpleasant sensory and emotional experience associated with, or resembling that associated with, actual or potential tissue damage. One type of longstanding pain is “chronic pain” that may be periodic or continuous and (1) persists beyond a normal recovery period or (2) occurs with a chronic health condition.

As used herein, the term “fiber” refers to an axon, which is a long slender projection of a nerve cell or neuron in vertebrate organisms having a diameter that corresponds to conduction velocity. Generally, a fiber conducts electrical impulses transmitting information in one or more directions throughout the body and is classified depending on the type of fiber (e.g., sensory, motor, etc.), the diameter of the fiber and/or if myelin coating is present.

As used herein, the term “nerve” refers to a bundle of fibers. For example, a nerve can be a sensory nerve that includes sensory fibers, a motor nerve that includes motor fibers, a sensorimotor nerve that includes sensory and motor fibers, etc.

As used herein, the term “sensory fiber(s)” refers to part of the peripheral nervous system (PNS) that conduct electrical impulses between a part of the body experiencing sensation and the brain/spinal cord. Sensory fibers have a range of fiber sizes. For example, sensory fibers can be classified as Aα (diameter 13-20 μm, conduction velocity 80-120 m/s, myelinated, associated with muscle spindle fibers and Golgi tendon organ); Aβ (diameter 6-12 μm, conduction velocity 33-75 m/s, myelinated, associated with all cutaneous mechanoreceptors); Aδ (diameter 1-5 μm conduction velocity 3-30 m/s, thinly myelinated, associated with free nerve endings of touch and pressure, nociceptors of the neospinothalamic tract, cold thermoreceptors); and C (diameter 0.2-1.5 μm, conduction velocity 0.5-2.0 m/s, unmyelinated, associated with nociceptors of the paleospinothalmic tract and warmth receptors).

As used herein, the term “motor fiber(s)” refer to part of the peripheral nervous system (PNS) that conduct electrical impulses between the brain/spinal cord and a part of the body. Motor fibers generally have a large fiber size. For example, motor fibers can be classified as Aα (diameter 13-20 μm, conduction velocity 89-120 m/s, myelinated, associated with extrafusal muscle fibers) or Aγ (diameter 5-8 μm, conduction velocity 4-24 m/s, myelinated, associated with intrafusal muscle fibers).

As used herein, the terms “photobiomodulation” and “PBM” refer to a form of light therapy based on the delivery of light with proper wavelengths to a patient by an emitter at a specific dosing scheme to achieve a desired response (or effect) at a target area. PBM utilizes non-ionizing light sources, including lasers, light emitting diodes, and/or broadband light sources and can be delivered by one or more emitters. In some examples, the light can have a wavelength between 250 nm and 1600 nm. However, as an example, the wavelength can be in the visible range (e.g., from 400 nm to 700 nm) and/or near-infrared range (e.g., from 700 nm to 1100 nm) of the electromagnetic spectrum.

As used herein, the term “direct PBM” refers to one or more doses of light configured by a controller (either external to the body or implanted at a location remote from the delivery) and/or external energy source and delivered from an internal emitter proximal or adjacent to at least one nerve target. For example, the emitter can be positioned such that intervening tissue has been removed between the emitter and the target nerve or neurovascular bundle (e.g., the emitter may be placed as much as 5 mm, 4 mm, 3 mm, 2 mm, 1 mm, 0.5 mm, 0.2 mm, 0.1 mm, about 0 mm, etc., from the nerve. In some instances, the emitter can be positioned with the intervening tissue no longer present (please note that an encapsulation layer may form around the emitter). In other instances, the emitter can be positioned with some of the intervening tissue removed (the encapsulation layer may form around the emitter).

As used herein, the term “internal” refers to something that is inside the body (e.g., near one or more nerves and/or one or more organs) and encapsulated by skin.

As used herein, the term “external” refers to something that is outside to the body.

As used herein, the terms “subject” and “patient” can be used interchangeably and refer to any warm-blooded vertebrate organism having a body, including, but not limited to, a human being, a pig, a rat, a mouse, a dog, a cat, a goat, a sheep, a horse, a monkey, an ape, a rabbit, a cow, etc.

### II. Overview

Chronic pain is often difficult to manage and can adversely affect all aspects of a sufferer's life if not treated and/or managed. Pharmaceutical treatments can be effective in many cases, but can have life altering, detrimental side effects, including addiction and overdose. Electrical nerve stimulation treatments, such as spinal cord stimulation (SCS) and peripheral nerve stimulation (and/or block) (PNS), have been explored as alternatives to pharmaceutical treatment, but have limited usefulness as electrical nerve stimulation treatments cannot be used to effectively treat all types of chronic pain. Photobiomodulation (PBM) is another avenue to provide non-pharmaceutical treatment of chronic pain in situations where SNS and PNS cannot be used. Lower dose transcutaneous PBM has been found to reduce pain in some cases, but the results have been mixed and the magnitude of the effect of transcutaneous PBM has been relatively modest, possibly due to the attenuation in the intervening tissue layers that reduces the amount of light reaching the target tissue. The presence of intervening tissues of various thicknesses and absorption properties, as well as many other factors, make it difficult to choose accurate and optimal parameters (e.g., wavelength(s), intensity, continuous wave versus pulsatile, spot size, etc.) for a transcutaneous PBM dose that is both safe and effective.

Direct application of PBM to a nerve can be used to provide a more accurate and effective therapeutic dose to the nerve to reduce pain. The nerve can be a sensory nerve and/or sensorimotor nerve that can include small diameter sensory fibers (e.g., C-fibers, A8-fibers, etc.) that carry pain signals. At least one dose of the direct PBM can be configured externally, transmitted to an internally implanted emitter, and then delivered directly to the nerve, without significant intervening tissue in the way. The at least one dose of the PBM directly applied to the nerve can inhibit conduction in at least one small diameter nerve fiber within the nerve without affecting conduction (or minimally affecting conduction) of at least one larger diameter nerve fiber within the nerve. Direct application of the at least one dose of PBM for the time set forth in the dose selectively inhibits at least a portion of conduction of at least one sensory signal by the at least one small diameter nerve fiber for another time (e.g., at least a day longer than the time).

### III. System

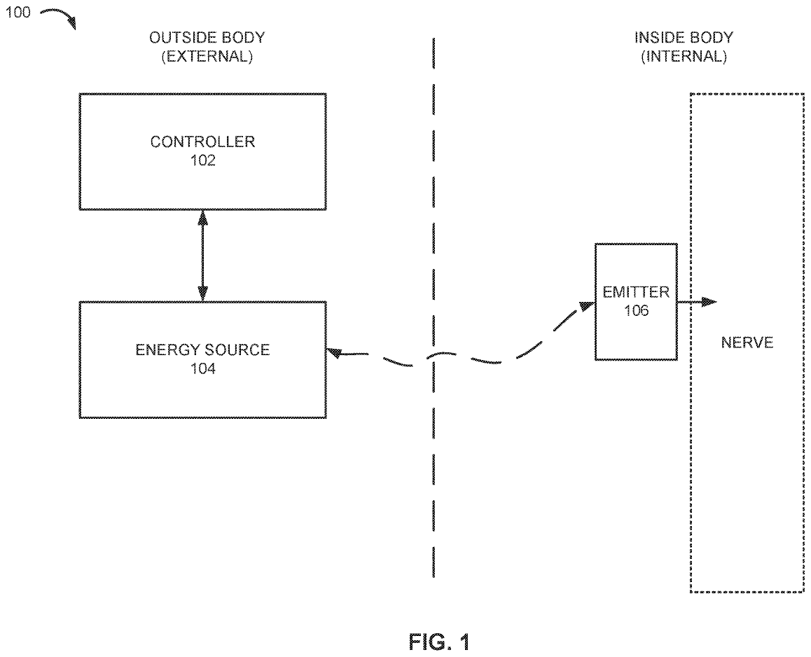

Provided herein is a system 100 (FIG. 1) for treatment of chronic pain via direct application of photobiomodulation (PBM) to a nerve. The PBM can be delivered directly to the nerve to block conduction in small fibers (e.g., fibers with a smaller diameter, such as C-fibers or Aδ-fibers) while allowing conduction in other fibers (e.g., motor fibers and larger sensory fibers, such as Aα, Aβ or some Aδ fibers). Direct application of PBM is superior to transcutaneous application of PBM for several reasons, including safer and more effective therapy and easier determination of accurate and effective parameters, because the light signal is not dissipated by skin, fat, muscle, or other tissues before reaching the target location. Additionally, internal components can deliver the PBM (otherwise referred to as a light signal of PBM) using reduced power demand compared to traditional transcutaneous application of PBM while maintaining similar, and/or better, pain relief.

The system 100 can include one or more external component (located outside the patient's body), such as controller 102 and energy source 104 shown in FIG. 1 that can configure one or more doses of PBM from outside the patient's body. It should be understood that additional external components (not shown) could also be included. The system 100 can also include one or more internal components (located inside the patient's body), shown as emitter 106 in FIG. 1 to deliver the configured one or more doses of PBM to one or more fibers within one or more nerves (represented as a single nerve) within the patient's body. It will be appreciated that additional internal components, such as one or more additional emitters, attachment mechanisms, wires, etc. (not shown) could also be included. The emitter 106 can be positioned near and/or adjacent to a target location on a nerve with no intervening tissue between the emitter and the nerve. It should be understood that while only a single emitter 106 is shown, one or more emitters can be positioned at one or more target locations on one or more nerves and can each be connected to the energy source 104. A dashed vertical line is illustrated to show the clear delineation between internal and external components (e.g., outside or inside of the skin of the patient). It should be understood that in some instances the controller 102 and/or the energy source may be implanted within the patient's body at one or more locations remote from the internal components,

The external components, such as controller 102 and energy source 104, can be maintained outside the patient's body for improved battery life of the system overall, easier controllability, reusability (can be coupled with multiple emitters for multiple patients), cost savings, or the like. It is important that the external components can be housed/used at a location (e.g., a physician's office, at home, at a clinic, etc.) and not within the patient. The internal components can be small and implanted within the patient so the patient can have mobility to go about everyday life without carrying around a bulky and/or visible medical device. As an example, application of the PBM treatment can be an outpatient procedure for the patient where the external components can be temporarily coupled to the internal components for a weekly, monthly, etc. procedure.

As noted, the external components can include at least a controller 102 and an energy source 104. Although illustrated as separate components, it should be understood that the energy source 104 can be a part of the controller 102 and/or can both be housed in a common device in some instances. The controller 102 and the energy source 104 can be connected with a wireless and/or a wired connection. The controller 102 can include a non-transitory memory and a processor (not illustrated in FIG. 1) that can be used to configure the one or more doses of PBM for treating the pain of the patient with one or more parameters (e.g., wavelength(s), power, intensity, duration, dosing scheme (including continuous wave versus pulsatile, timings, etc.), spot size, duty cycle, and the like). As an example, the memory and processor can be embodied as a microprocessor. The parameters of the dose of PBM can be configured based on at least one of the type of pain, the location of the target nerve and/or the pain, the type of target fiber(s), the amount of relief, the length of relief, the patient's body conditions (e.g., health, weight, age, gender, muscle mass, etc.), and/or conditions of one or more similar patients. For instance, the parameters of the dose of PBM can be retrieved from a preset list of parameters based on a characteristic or condition of the patient (e.g., severity of pain, additional/corresponding medical conditions, age, etc.). In some instances, the controller 102 can receive one or more inputs related to the one or more doses of PBM for the patient. The one or more inputs can be manual inputs from a medical professional (e.g., related to pain relief effectiveness of a dose of PBM and/or several doses of PBM over time, discomfort of the patient, etc.) or automatic inputs (e.g., based on one or more sensors measuring downstream/upstream neural conduction, pain of the patient, mobility of the patient, temperature of the nerve and/or surrounding tissues, etc.) The energy source 104 (also referred to as an electrical energy source) can deliver power to the emitter 106 to power on the emitter to form and deliver the one or more dose of light associated with PBM. The energy source 104 can be in electrical communication with the internal components such as emitter 106. The electrical communication can be wireless and/or wired (e.g., percutaneous wire connecting the emitter 106 and energy source 104).

The internal components can include at least the emitter 106 (also referred to as the “implantable” emitter) and any components (not illustrated) that are related to operation of the emitter 106. The emitter 106 can be configured to communicate with the energy source 104 and/or the controller 102 according to a wired and/or wireless signal (illustrated as wireless communication between the energy source 104 and the emitter 106, but the illustration is not meant to be limiting). The connection can be temporary (e.g., only for the time the treatment procedure takes). For instance, the emitter 106 can be connected to a connection component (e.g., a connector, a first side of a plug, etc.) that can be configured to connect with a connection component (e.g., a connector, a second side of a plug, etc.) of the external component(s) (the controller 102 and/or the energy source 104). In another instance, the emitter 106 can include a wireless communication component (not shown, but an example of the wireless communication component can facilitate RF communication, Bluetooth communication, or the like) to engage in wireless communication with the external component(s) (the controller 102 and/or the energy source 104) also having a wireless communication component (not shown). The controller 102 can send the one or more parameters for the configured PBM (e.g., the light signal) to be applied to the emitter 106 and/or to the energy source 104 and the energy source can send power to the emitter. Notably, the emitter 106, in some instances, does not have an independent power source and requires the power from the energy source 104. The emitter 106 can deliver the light signal of the PBM to the nerve according to the configuration (e.g., the one or more parameters) received from the controller 102. As an example, the emitter 106 can be a laser diode connected to an optical fiber driven by a laser diode driver (the controller 102 and energy source 104) through a thermoelectrically controlled diode mount.

As noted, during a treatment, the external components (e.g., the energy source 104 and/or the controller 102) can be temporarily coupled to the emitter 106 for a time period. As an example, the time period can be at most the time period required to drive delivery of at least one dose of PBM from the emitter 106 directly to at least one nerve. In another example, the time period can be only the time needed to deliver the necessary power and one or more parameters to the emitter 106, which can be less than the time period required to drive delivery of the at least one dose of PBM. The time period can be, for example, an hour or less, 12 minutes or less, 10 minutes or less, 5 minutes or less, 1 minute or less, 30 seconds or less, or the like. Optionally, the controller 102 can include at least one notification device (e.g., screen, speaker, haptic motor, etc.) and can notify the medical professional when the time period is complete and the controller and/or energy source 104 and the emitter 106 can be decoupled.

The emitter 106 can be located near and/or adjacent (e.g., 5 mm or less, 4 mm or less, 3 mm or less, 2 mm or less, 1 mm or less, or the like) to one or more sensory nerves (including only sensory fibers), one or more motor nerves (including only motor fibers), one or more sensorimotor nerves (including a mix of sensory and motor fibers), or the like. While not shown, one or more emitters can be positioned near and/or adjacent the same nerve (at one or more target locations) and/or one or more emitters can be positioned near and/or adjacent different nerves (in the same or different parts of the patient's body). It should be understood that any description for one emitter 106 can apply to one or more emitters. The emitter 106 can directly deliver (no intervening tissue) the at least one dose of PBM and the nerve (e.g., the sensory nerve, the motor nerve, or the sensorimotor nerve) can directly receive the PBM signal (also referred to as “the light signal of PBM”, “PBM light signal”, and the like).

The direct application of the PBM signal from the emitter 106 to the nerve can block one or more fibers in the nerve. The emitter 106 can directly apply the PBM signal for a time, which can be the same as or different from the time period for driving the delivery and/or transferring power and parameters to the emitter 106 from the controller 102 and/or energy source 104). The PBM signal can be directly applied to at least one sensory and/or sensorimotor nerve to selectively inhibit conduction in at least one small diameter nerve fiber without affecting, or minimally affecting, conduction of at least one larger diameter nerve fiber in the same nerve. For instance, the PBM light signal can be configured (by the controller 102) and directly applied (by the emitter 106) to the nerve to block conduction of pain signals in small diameter sensory fibers within the nerve without affecting conduction of at least one larger diameter nerve fiber in the at least one sensory and/or sensorimotor nerve. Conduction of the at least one sensory signal can be inhibited for another time that can be at least a day longer than the time the light signal was delivered by the emitter 106. The small diameter sensory fiber(s) within the nerve can include sensory fibers operational for the transport of pain signals (e.g., C-fibers, Aδ-fibers, or the like). For example, the small diameter sensory fiber(s) can be nociceptors.

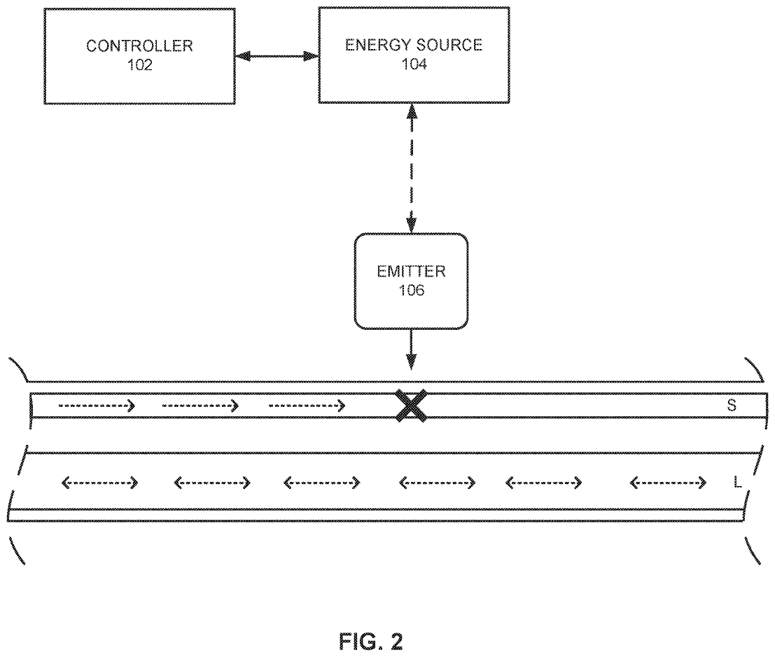

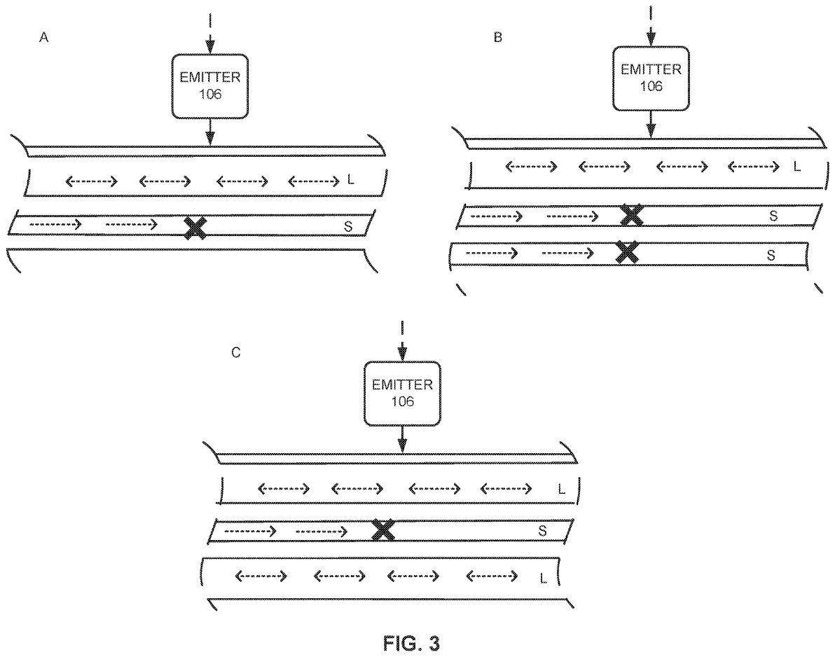

FIGS. 2 and 3 show examples of using the system of FIG. 1 to apply light to block conduction in at least one small diameter nerve fiber in a nerve while not affecting, or minimally affecting, conduction in at least one larger diameter nerve fiber. As illustrated, conduction in large nerve fibers can be either an upstream or downstream directions, such that the large nerve fibers can be motor fibers (conducting mechanical signals to muscles) and/or large sensory fibers (e.g., Aα, Aβ, or some Aδ) that are larger than C fibers and/or Aδ fibers, that can conduct sensory signals other than pain or motor fibers (Aα and/or Aγ) that conduct motor signals). It should be understood that the nerve can have multiple nerve fibers (of varying sizes) that can each conduct in different directions; the two-headed arrows are shown in FIGS. 2 and 3 to signify uni-directional conduction in either direction in one nerve (depending on nerve type and location) and/or, if physiologically possible, bi-directional conduction in a single fiber.

As shown in FIGS. 2 and 3, the controller 102 can configure at least one dose of PBM, having one or more parameters and then communicate that configuration to the energy source 104. Then the energy source 104 can communicate the configuration of the at least one dose of PBM and the energy necessary to delivery that at least one dose of PBM to the emitter 106. The emitter 106 can be located near and/or adjacent the nerve when the intervening tissue between the emitter and the nerve has been removed (but the emitter 106 may be encapsulated post implant) to directly apply the at least one dose of PBM. The light signal of the at least one dose of PBM can block conduction (represented as an X) in one or more of the small diameter fibers (e.g., C-fibers and/or Aδ-fibers) for the time of application and a carry over time at least one day longer than the time of application. Conduction in the at least one small diameter fiber can be blocked while not affecting, or minimally affecting, conduction in at least one larger diameter nerve fiber. In FIG. 2, the nerve is illustrated as including a single small diameter fiber and a large diameter fiber with the small diameter fiber closer to the emitter. The directly applied dose of PBM blocks conduction in the small diameter fiber while not affecting conduction in the larger diameter fiber. For example, chronic pain could be blocked while other acute sensations can be felt, or pain can be blocked and motor abilities can be unaffected. While not wishing to be bound by theory, the positioning of the emitter relative to where small and large diameter fibers are in the nerve bundle does not affect the use of the system. Accordingly, FIG. 3, element A illustrates the emitter 106 being positioned nearer a larger diameter nerve than a smaller diameter nerve and blocking conduction in the smaller diameter nerve but not the larger diameter nerve. FIG. 3, element B illustrates the emitter 106 positioned relative a nerve with two smaller diameter fibers and one larger diameter fiber, where conduction in both smaller diameter fibers are blocked and conduction in the larger diameter fiber is not. FIG. 3, element C illustrate the emitter 106 positioned relative a nerve with a smaller diameter fiber between two larger diameter fibers and conduction in both larger diameter fibers is not affected while conduction in the middle smaller diameter fiber is blocked. It should be understood that each sensory and/or sensorimotor nerve can hold any number of smaller and/or larger diameter fibers and FIGS. 2 and 3 are merely example illustrations.

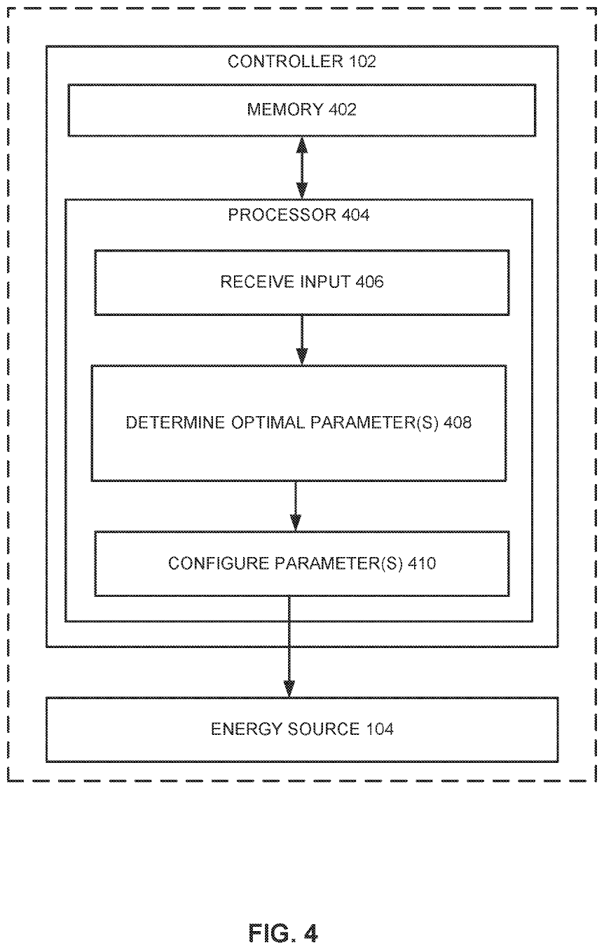

The controller 102, as shown in FIG. 1, can program one or more parameters of the PBM light signal and the energy source 104. The energy source 104, as shown in FIG. 1, can configure an energy signal to power the emitter 106. FIG. 4 shows an example of the controller 102 and energy source 104 embodied within a single device. The controller 102 can include a memory 402 and a processor 404, which could be within a single device like a microprocessor. The memory 402 can be a non-transitory memory and can store one or more instructions. The processor 404 can access the memory 402 to execute the instructions, including receive an input 406 (which may be a preprogrammed input or an input from a user) related to applying at least one dose of PBM directly to a nerve of a patient, determine one or more optimal parameters 408 for the at least one dose of PBM, and configure one or more parameters 410 for the PBM delivered to the patient. The configuration can be sent to the energy source 104 and then the energy and the configuration can be sent to the emitter (not shown in FIG. 4).

The controller 102 and the energy source 104 can configure the dose and the energy signal to be sent to the emitter 104 based on the one or more parameters. The one or more parameters can include wavelength(s) of the light signal, power of the energy source, intensity of the light signal, duration of application, dosing scheme (including continuous wave versus pulsatile, number of dosages to be applied in a given time, or the like), maximum temperature threshold of the nerve, spot size of the light signal, timing of the application (e.g., pulses, mixing different wavelengths and/or intensities or the like), etc.), duty cycle, and the like. After the time to drive delivery of the application and/or transfer the energy and/or the parameters to deliver the application of the PBM light signal, the connection between the external components and the internal components can be removed.

The at least one dose of PBM is applied for a given duration (e.g., time) to selectively inhibit at least a portion of conduction of the at least one small diameter sensory fiber within the nerve while allowing conduction in larger diameter fibers. The block of conduction of the at least one small diameter sensory fiber carries over for another time significantly longer than the time of application (e.g., extends for a time period longer than the application of the PBM light signal) without affecting conduction of the at least one larger diameter fiber within the nerve. For example, the time period of application can be an hour or less (e.g., the time period can be shorter including 45 minutes or less, 30 minutes or less, 20 minutes or less, 15 minutes or less, or the like) and the other time period (the carryover time of the effect from the specific direct PBM configuration) can be at least a day (e.g., the other time can extend for at least 2 days, at least 3 days, at least 4 days, at least 5 days, at least 10 days, at least 15 days, at least 20 days, at least 25 days, at least 50 days, or more).

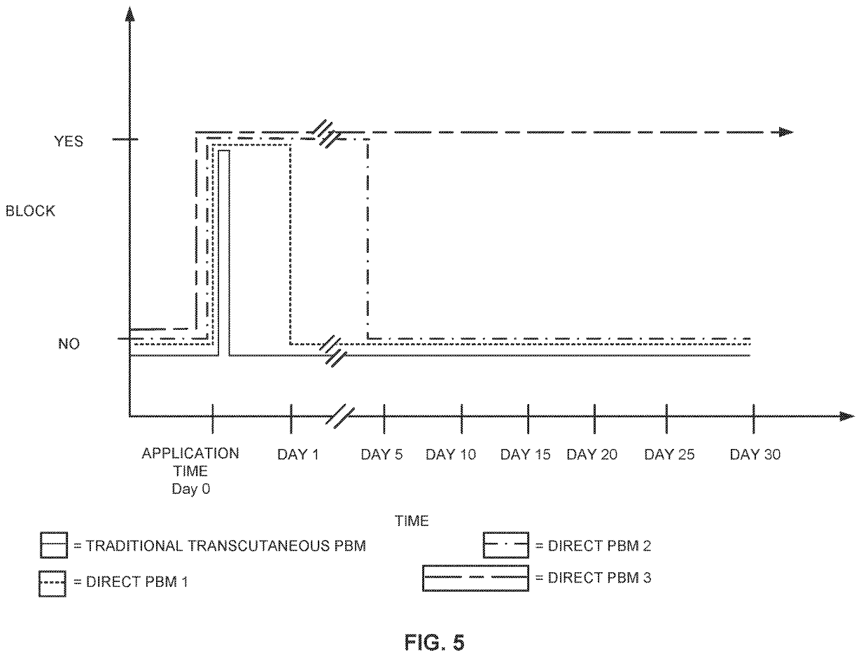

FIG. 5 shows an example plot of the time course of different PBM blocks for comparison purposes. Traditional transcutaneous PBM (continuous line) is effective for the time of application, with only a small amount of carryover effect-less than a day, likely (but not wishing to be bound by theory) due to the PBM light signal being dispersed and/or absorbed by tissues not the nerve. Different configurations of direct PBM are illustrates as direct PBM 1 (dotted line), direct PBM 2 (short dash, dot, short dash line), and direct PBM 3 (long dash, short dash, long dash line). Each can be configured with at least one different parameter and can have an effect of block lasting far beyond the application time (e.g., one day, five days, or 30 days or longer as shown).

### IV. Method

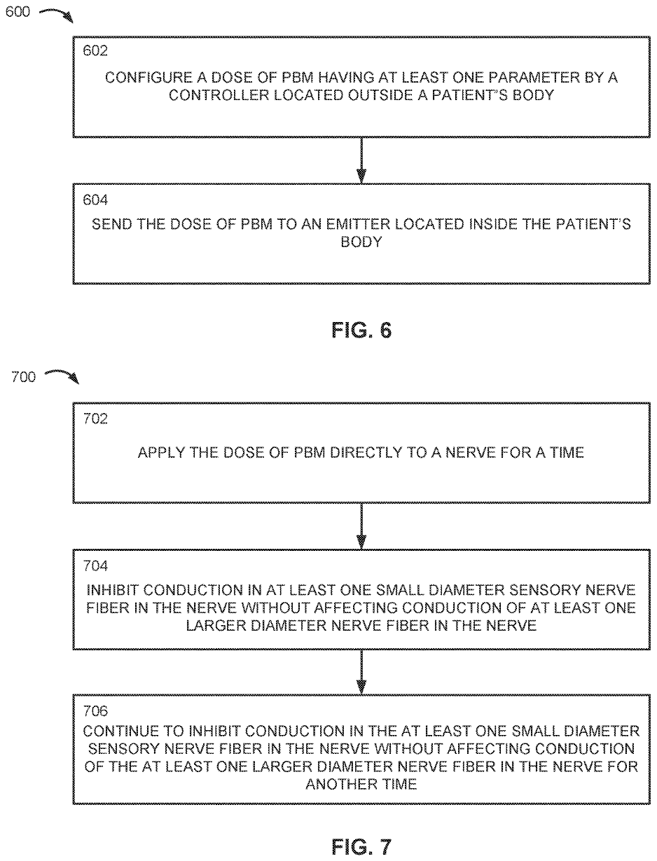

Another aspect of the present disclosure can include methods 600, 700, and 800 (FIGS. 6-8) for treatment of chronic pain via direct application of PBM to a nerve. The methods 600, 700, and 800 can be performed by the system 100 of FIG. 1 (whose use is detailed further with regard to FIGS. 2-4). The methods 600, 700, and 800 are illustrated as process flow diagrams with flowchart illustrations that can be implemented by one or more components of the system 100. For purposes of simplicity, the methods 600, 700, and 800 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 600, 700, and 800.

FIG. 6 shows an example method 600 performed by the external components (e.g., controller 102 and energy source 104, which may be in a common device) of FIG. 1 for providing a dose of PBM that can be used to treat a patient's sensation of pain (e.g., inhibiting at least one sensory signal related nociception and/or to chronic pain of the patient). At 602, a dose of PBM having at least one parameter can be configured by a controller outside of the patient's body. The dose can be configured by determining and configuring at least one parameter of the dose of PBM (e.g., a power, a wavelength, a duration, a dosing scheme, a duty cycle density, an intensity, a timing, a number of doses, a type of waveform, or the like). The parameter of the dose of PBM can be configured based on at least one of the patient's body conditions (e.g., health, weight, age, gender, muscle mass, etc.), and/or conditions of one or more similar patients, one or more other patients with the same condition, body weight, age, etc., and/or the light. For instance, the parameters of the dose of PBM can be retrieved from a preset list of parameters based on a characteristic of the patient (e.g., severity of pain, additional/corresponding medical conditions, age, etc.). In another instance, the parameters can be input by a medical professional. At 604, the dose of PBM can be sent, by the controller and/or the energy source, to an emitter located inside the patient's body. For example, the emitter can be implanted proximal to (or adjacent to) a nerve target. The nerve target can be at least one sensory and/or sensorimotor nerve of the patient. For example, the emitter can be positioned such that there is no intervening tissue between the emitter and the target nerve (e.g., the emitter may be placed as much as 5 mm, 4 mm, 3 mm, 2 mm, 1 mm, 0.5 mm, 0.2 mm, 0.1 mm, ~0 mm, etc., from the nerve with no intervening tissue present).

FIG. 7 shows an example method 700 performed at least in part by the internal components (emitter 106) of FIG. 1 for applying a dose of PBM. At 702, the dose of PBM (configured by one or more external components and received by an internal emitter) can be applied directly to a nerve (e.g., one or more nerves) for a time. For example, the time can be less than an hour, less than 30 minutes, less than 15 minutes, less than 12 minutes, or the like. At 704, the dose of PBM can inhibit conduction in at least one small diameter sensory nerve fiber (e.g., S in FIGS. 2-3) in the nerve without affecting conduction of at least one larger diameter nerve fiber (e.g., L in FIGS. 2-3) in the nerve. In other words, application of the dose of PBM directly to the at least one sensory and/or sensorimotor nerve for the time to selectively inhibit conduction in at least one small diameter sensory nerve fiber in the at least one sensory and/or sensorimotor nerve without affecting conduction of at least one larger diameter nerve fiber in the at least one sensory and/or sensorimotor nerve. Notably, the power of the PBM can be lower than a power required for transcutaneous PBM to inhibit conduction of at least one sensory signal. The application of the PBM, as an example, to at least one sensorimotor nerve allows at least one mechanical signal to conduct through the at least one larger diameter nerve fiber in the at least one sensorimotor nerve normally while conduction in a sensory fiber is blocked. At 706, the inhibition of the conduction in the at least one small diameter sensory nerve fiber (e.g., S in FIGS. 2-3) in the nerve without affecting conduction of at least one larger diameter nerve fiber (e.g., L in FIGS. 2-3) in the nerve for another time longer than the time of application. In other words, conduction of the at least one sensory signal is inhibited for another time (at least a day, three days, five days, a week, two weeks, three weeks, a month (30 days) or more) longer than the time of application.

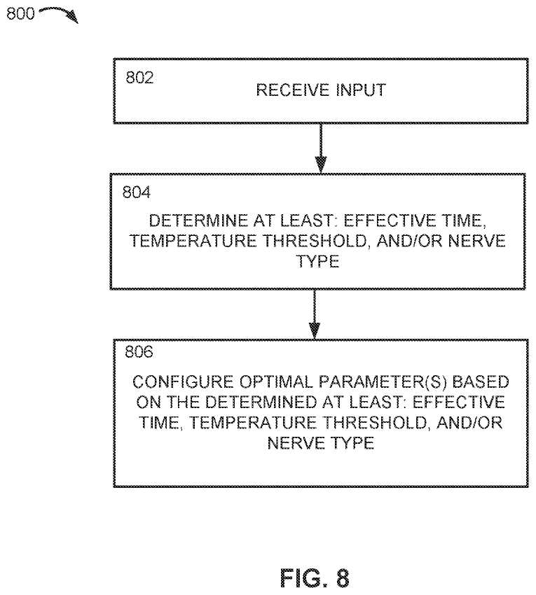

FIG. 8 shows an example method 800 performed by an external component (e.g., at least controller 102) of FIG. 1 to configure parameters for the PBM. At 802, an input can be received (e.g., from the patient, from an individual remote from the patient, from a source related to the controller 102, etc.). At 804, based on the input, at least an effective time, temperature threshold, nerve type, etc. (any parameter based on the nerve, the patient, the PBM, or the like) can be determined. At 806, one or more optimal parameter(s) can be configured based on the determined at least effective time, temperature threshold, nerve type, etc.

### V. Experimental

Described herein is a study that applies photobiomodulation (PBM) directly to one or more nerves and measures the effects of the PBM on small fibers and large fibers within the one or more nerves over an extended period. The study revealed that the direct application of the PBM yields effects consistent with inhibition of conduction in the small fibers over the extended period without affecting the conduction in the one or more large fibers.

### Methods

All studies were performed in accordance with the NIH Guide for Care and Use of Laboratory Animals (NIH Publication No. 80-23) revised in 1996, and in compliance with ARRIVE (Animal Research: Reporting of In Vivo Experiments) guidelines (http://www.nc3rs.org.uk/page.asp? id=1357). Experimental methods involving animals were reviewed and approved by the Case Western Reserve University (CWRU) Institutional Animal Care and Use Committee (IACUC).

### SNI Model Rationale

The Spared Nerve Injury (SNI) chronic pain model has been used extensively in rodent behavioral studies to determine the effects of various candidate treatments including pharmacological therapeutics, spinal cord stimulation, and PBM. The SNI pain model is generated by ligating and transecting the tibial and peroneal branches of the sciatic nerve while leaving the sural nerve intact and is characterized by prolonged thermal and mechanical hypersensitivity that reaches a chronic steady state 5 to 7 days after transection. Following SNI, thermal hypersensitivity is expected to be mediated by excitation of smaller unmyelinated fibers, and mechanical hypersensitivity is expected to be mediated by excitation of larger myelinated sensory fibers. Data from previous transcutaneous PBM investigations using inhibitory doses showed that PBM can reduce thermal hypersensitivity (presumably mediated by effects on smaller diameter fibers), but that large-fiber mechanical hypersensitivity remained unchanged following PBM (presumably because large cutaneous fibers are unaffected). Here, a single application of PBM 1 mm away from the sural nerve was delivered on day 7 post SNI surgery. The behavioral responses (small-fiber thermal sensitivity—Hargreave's test; large-fiber mechanical sensitivity—von Frey evaluation) were measured over a two-week period. The application of PBM with no tissue between the nerve and emitter enables robust assessment of dose on behavioral effects.

### Animals

Ninety-one Sprague-Dawley rats (200-300 g, Charles River Laboratories International, Inc.) were used in this study. The rats were housed alone under a 12-hour light/12-hour dark schedule, with free access to food and water. Animals were divided into two control groups and five experimental groups (beginning with 6 male and 7 females per group). The control groups included: (1) negative control: No SNI surgery, no PBM, and (2) Positive control: SNI surgery, no PBM. All rats underwent an initial SNI surgery and a second surgery to expose the sural nerve to deliver light (see Table 1). Two animals died unrelated to the surgical procedures and PBM application.

TABLE 1
Description of experimental groups
with number of animals in each group.

| | | Number |
|---|---|---|
| Group | | of Animals |
| 1 | Negative Control (SNI−/PBM−) | 13 |
| 2 | Positive Control (SNI+/PBM−) | 13 |
| 3 | PBM Low Power/High Energy: SNI+/ | 13 (1 died |
| | PBM = 20 mW for 12 minutes | unrelated |
| | | to PBM |
| | | application) |
| 4 | PBM High Power/High Energy: SNI+/ | 13 |
| | PBM = 60 mW for 4 minutes | |
| 5 | PBM Low Power/Low Energy: SNI+/ | 13 |
| | PBM = 20 mW for 4 minutes | |
| 6 | PBM High Power/Low Energy: SNI+/ | 13 (1 died |
| | PBM = 60 mW for 80 seconds | unrelated |
| | | to PBM |
| | | application) |
| 7 | PBM of Healthy: SNI−/ | 13 |
| | PBM = 60 mW for 4 minutes | |

| | |
|---|---|
| Total | 91 |

### Surgery—Spared Nerve Injury (SNI)

SNI (or sham) surgeries were performed on Day 1 in all groups to generate chronic hypersensitivities. Rats were anesthetized with isoflurane (2-4%). A small incision was made on the lateral left thigh through the bicep femoris to expose the trifurcation of the sciatic nerve. The common peroneal and tibial branches were tightly ligated with two sutures each, and a 3-5 mm intervening portion of each nerve was transected and discarded. Disruption of the sural nerve was carefully avoided during the surgical process. The muscle layers were sutured, and the skin was closed using wound clips. Groups that were in the sham SNI cohorts received the identical surgery, but without the ligation and transection.

### Surgery—Photobiomodulation Treatment and PBM Groups

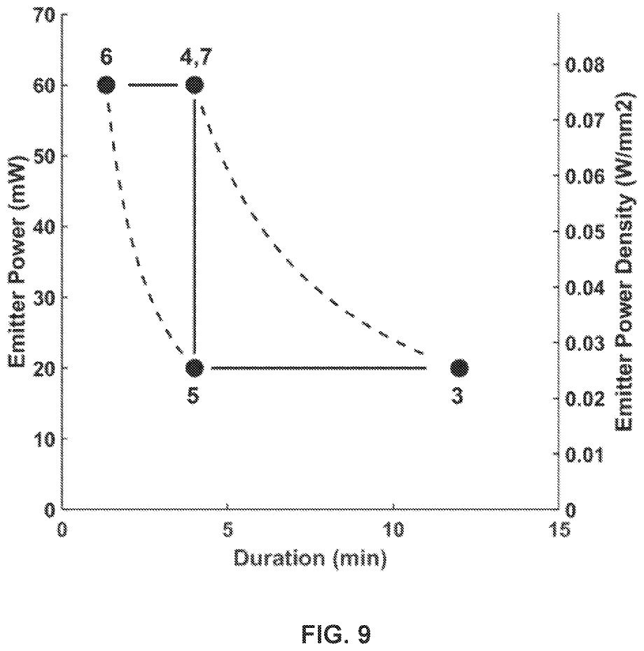

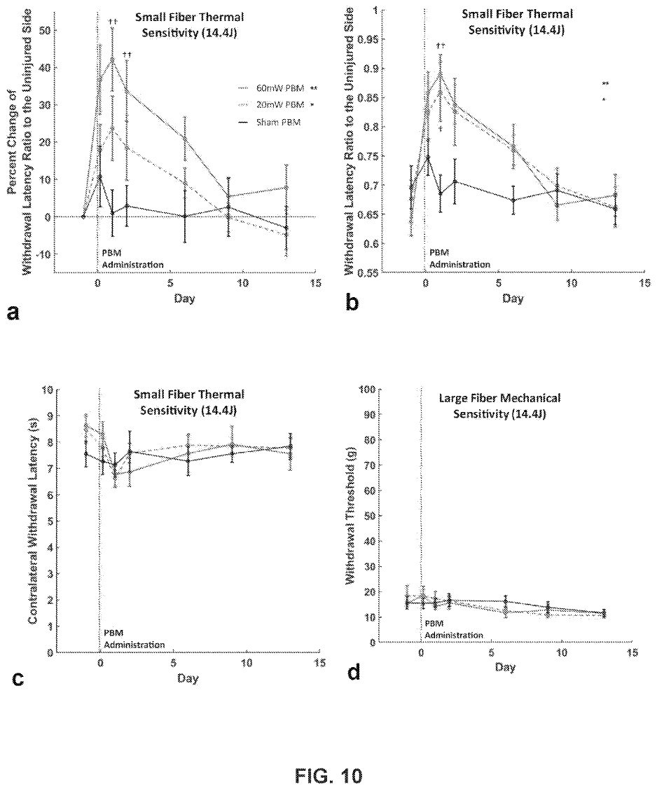

PBM (or sham) was applied to the sural nerve in multiple experimental groups on Day 8 during a second surgical intervention. For those rats in sham PBM groups, the same surgical and procedural steps described below were taken but without turning on the laser. Rats were anesthetized in the same manner as used during the SNI surgery. The incision site was reopened, and the trifurcation point exposed. Fascia around the sural nerve was carefully removed, taking great care to avoid disturbance of the nerve itself. A fiber optic emitter was inserted into the cavity directly above the sural nerve. Using a micromanipulator, the tip of the fiber optic emitter was lowered to gently touch the nerve, then lifted 1.0 mm away. Two groups did not receive PBM treatment but had the surgical intervention: (1) sham SNI+sham PBM, and (2) SNI+sham PBM. PBM was applied using specific parameters to the SNI+PBM groups: (3) 20 mW for 12 minutes, (4) 60 mW for 4 minutes, (5) 20 mW for 4 minutes and (6) 60 mW for 1.33 minutes (80 sec) (see FIG. 9 and Table 1). A final group (7) had a sham SNI surgery with PBM parameters of 60 mW for 4 minutes. The spot size of the emitter was 0.00785 cm² (1 mm diameter) and yielded power densities of 2.55 W/cm² and 7.64 W/cm² for 20 mW and 60 mW groups, respectively, and given the 1 mm distance to the nerve and a divergence angle of 22.95°, the spot size at the tissue is about 1.85 mm diameter, yielding a spot area of 2.69 mm². The calculated power densities at the tissue are then 2.23 W/cm² and 0.743 W/cm². The calculated energy densities were 1834 J/cm², 611 J/cm² at the emitter, and 536 J/cm², 179 J/cm² at the tissue. Upon completion of light delivery, the muscle layers were sutured, and the skin was closed using 2-3 wound clips.

In FIG. 9, the power of the emitter and the duration of PBM application are plotted for the four treatment groups tested on the SNI pain model. The solid vertical line highlights the same duration of application used by groups 3 and 4; the solid horizontal lines highlight the same emitter power used by groups 3 and 5, and by groups 4 and 6; the dashed lines indicate same total energy density delivered from the emitter (611 J/cm² in groups 3 and 6, and 1834 J/cm² in groups 4 and 5). Group 7 used the same power and duration combination as group 4 but had the sham SNI surgery.

### Laser Irradiation System

PBM was applied in groups 3-6, as mentioned previously. An 808 nm wavelength laser diode (model LDX-3410-808-HHL-105 by RPMC, O'Fallon, MO) was driven by a laser diode driver (model ILX LDC3736 by Newport, Andover, MA) through a thermoelectrically controlled diode mount (model 244HHL by Arroyo Instruments, San Luis Obispo, CA). Light was emitted from a 1 mm diameter optical fiber (model M35L02 by Thorlabs, Newton, NJ). The power of emission was calibrated before each surgical procedure (PowerMax, Coherent Scientific, Santa Clara, CA) and tested again afterwards to ensure the maintenance of the power.

### Behavioral Tests—Thermal (Heat) Hypersensitivity (Hargreave's Test)

The Hargreave's test procedure was as follows. Rats were placed on an elevated, temperature-controlled surface (IITC, Woodland Hills, CA) and allowed to acclimate for 5-10 minutes. The heat beam (focused 4×6 mm beam spot) was placed on the lateral plantar surface of the hind paw (area of sural nerve innervation) and a withdrawal time was recorded. To ensure that no damage to the paw occurred, the maximum heat application duration was pre-programmed to 20 seconds. Both the ipsilateral (spared-nerve injury) and contralateral (uninjured) sides were tested. The rats were tested five times with 5 minutes between each test, and the highest and lowest values were discarded. An average response-time ratio between ipsilateral and contralateral sides was calculated. This test was used to measure behavioral hypersensitivity expected to be mediated by activity of smaller diameter fibers (C- and Aδ-fibers).

### Behavioral Tests—Mechanical Hypersensitivity (Von Frey Filaments)

The Von Frey test procedure was as follows. Rats were placed on an elevated, meshed grid surface and allowed to acclimate for 5-10 minutes (IITC, Woodland Hills, CA). Von Frey filaments (beginning at 6 g) were applied to the lateral plantar surface of the injured hind paw. Filaments of increasing force were applied until the paw was retracted in response to filament application (each filament was applied 5 times using the ascending stimulus method). The test was performed 5 times with at least 5 minutes between each test and the mean of the 5 measurements was calculated and presented. This evaluation was used to measure hypersensitivity mediated by large fibers (Aβ- and Aδ-fibers).

Determination of Temperature Increase with Direct Application of the Laser on the Nerve

Since the PBM power density is significantly higher than those of previous studies, surface temperature increases of the nerves were measured before, during, and after PBM application. In 4 separate experimental animals, PBM (60 mW for 4 minutes and 20 mW for 4 minutes) was applied to the sural nerve, and temperature changes were recorded via a thermal camera (model A655sc by FLIR Systems, Inc.).

### Statistical Analysis

All data are shown as the mean±SEM. Statistical comparisons of the hypersensitivity over time trajectories between treatment and control groups were made with a two-way ANOVA. For statistical analyses of day-to-day hypersensitivity data, a homoscedastic, two-tailed student's t-test with Dunn-Sidak's multiple comparisons test was used to compare treatment groups to the positive control. Family-wise significance and confidence intervals were set to 0.05 (with multiple comparisons test, the p-value was adjusted to 0.0073). All statistical values are presented in Tables 2 and 3.

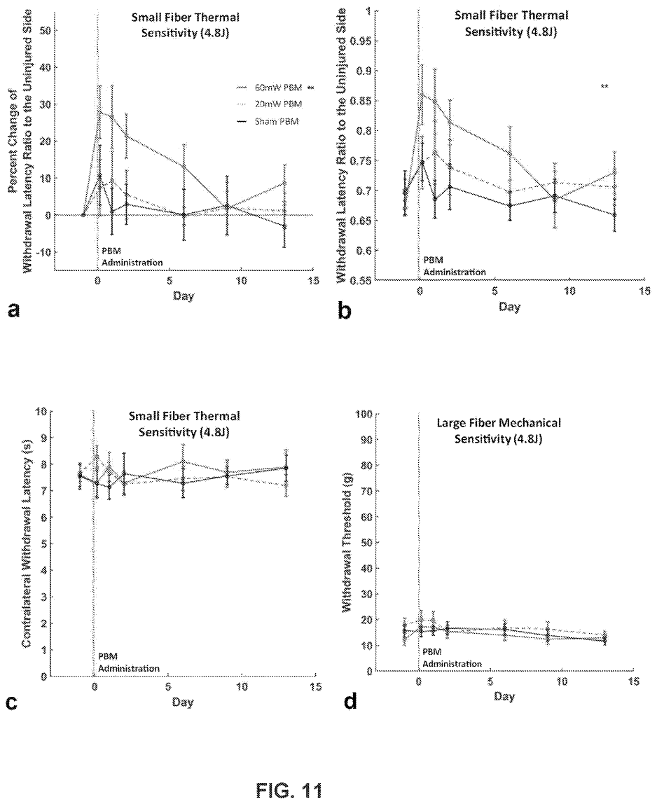

TABLE 2
P-values for the data corresponding to FIGS. 10, element a,
and 11, element a. Green represents a significant difference
from the positive control. Orange represents a trend
(0.0073 < P-value < 0.05) from positive control.
Red represents non-significance from the positive control.

| Day after | 60 mW, | 60 mW, | 20 mW, | 20 mW, |
|---|---|---|---|---|
| PBM | 4 min | 1.33 min | 12 min | 4 min |
| Application | (Group 4) | (Group 6) | (Group 3) | (Group 5) |
| −1 | N/A | N/A | N/A | N/A |
| 0.25 | 0.024164 | 0.084191 | 0.457204 | 0.734476 |
| | (Orange) | (Red) | (Red) | (Red) |
| 1 | 0.000156 | 0.009633 | 0.020323 | 0.369545 |
| | (Green) | (Orange) | (Orange) | (Red) |
| 2 | 0.001477 | 0.014854 | 0.088426 | 0.730881 |
| | (Green) | (Orange) | (Red) | (Red) |
| 6 | 0.014264 | 0.120185 | 0.22218 | 0.949728 |
| | (Orange) | (Red) | (Red) | (Red) |
| 9 | 0.725386 | 0.928602 | 0.726059 | 0.932561 |
| | (Red) | (Red) | (Red) | (Red) |
| 13 | 0.151636 | 0.093374 | 0.793674 | 0.535054 |
| | (Red) | (Red) | (Red) | (Red) |

TABLE 3
P-values for the data corresponding to FIGS. 10, element b,
and 11, element b. Green represents a significant difference
from the positive control. Orange represents a trend
(0.0073 < P-value < 0.05) from positive control.
Red represents non-significance from the positive control.

| Day after | 60 mW, | 60 mW, | 20 mW, | 20 mW, |
|---|---|---|---|---|
| PBM | 4 min | 1.33 min | 12 min | 4 min |
| Application | (Group 4) | (Group 6) | (Group 3) | (Group 5) |
| −1 | 0.187512 | 0.535067 | 0.697059 | 0.909319 |
| | (Red) | (Red) | (Red) | (Red) |
| 0.25 | 0.030163 | 0.063088 | 0.188896 | 0.986931 |
| | (Orange) | (Red) | (Red) | (Red) |
| 1 | 0.000156 | 0.013809 | 0.006676 | 0.214056 |
| | (Green) | (Orange) | (Green) | (Red) |
| 2 | 0.015661 | 0.05776 | 0.091477 | 0.589807 |
| | (Orange) | (Red) | (Red) | (Red) |
| 6 | 0.05158 | 0.090423 | 0.023473 | 0.461717 |
| | (Red) | (Red) | (Orange) | (Red) |
| 9 | 0.51117 | 0.908223 | 0.856492 | 0.605933 |
| | (Red) | (Red) | (Red) | (Red) |
| 13 | 0.604046 | 0.117431 | 0.94078 | 0.2539 |
| | (Red) | (Red) | Red) | (Red) |

It should be noted that FIG. 10, elements a and b show that high energy PBM reduces hypersensitivities associated with the small-diameter nerve fibers, with FIG. 10, element a, illustrating how reduction of withdrawal latency ratio for individual rats is increased by over 20% for both the high and low power groups and FIG. 10, element b illustrating that the raw withdrawal latency ratio is significantly increased from the sham PBM group. Similarly, FIG. 11, elements a and b show that low energy PBM reduces hypersensitivities associated with the small diameter nerve fibers when higher power (60 mW) is used with FIG. 11, element a showing a reduction of withdrawal latency ratio for individual rats is increased by over 20% for the high, but not the low power group and FIG. 11, element b shows that the raw withdrawal latency ratio is significantly increased from the sham PBM group for the high power group.

### Results

### Effects of PBM Doses at the Nerve

PBM applied at the higher total energy (14.4 J) For each animal, the data were normalized to the withdrawal time ratio measured in that animal the day prior to PBM administration, plotted as percent change in withdrawal time ratio (see FIG. 10, element a), and statistically compared to the positive control (group 2). The statistical analysis revealed a significant rise in withdrawal time ratios in PBM groups 3 and 4 compared to the control group. The data of FIG. 10, element a suggests that there may be a difference between the response magnitudes of group 3 and group 4, but no statistically significant difference was found. PBM applied at the higher total energy (14.4 J) using both 20 and 60 mW (for 12 minutes or for 4 minutes, respectively) yielded thermal stimulus induced withdrawal ratios (not normalized) with similar magnitude and time course (see FIG. 10, element b). PBM increased the withdrawal time ratio (injured side/uninjured side) from approximately 0.67 to (0.85-0.88) at the measurement day of peak response (typically one day after PBM application). The withdrawal time ratios returned to SNI baseline levels about 7 to 10 days following PBM application. Of note, the response latencies of the healthy (contralateral) paw were stable throughout the experiment (see FIG. 10, element c, showing the contralateral and sham PBM control withdrawal latencies remain stable throughout the experiment), and neither PBM dosing scheme that used high energy (14.4 J) resulted in detectable changes to mechanical hypersensitivity (see FIG. 10, element d), showing mechanical allodynia is unchanged with respect to treatment differences via 2-way ANOVA).

PBM applied at the lower total energy (4.8 J) PBM application for group 6 (60 mW for 1.33 minutes) led to comparable responses in magnitude and time course as the two high-energy doses (compare FIG. 10, elements a and b, to FIG. 11, elements a and b) and was statistically different than the control. PBM application for group 5 (20 mW for 4 minutes) did not yield a statistically significant response. The response latencies of the healthy (contralateral) paw were stable throughout the experiment (see FIG. 11, element c), showing the contralateral PBM control withdrawal latency does not drop below the sham PBM control for either treatment group), and neither PBM application with a low energy dosing scheme (4.8 J) resulted in detectable changes to mechanical hypersensitivity (see FIG. 11, element d), showing mechanical allodynia is unchanged with respect to treatment differences via 2-way ANOVA).

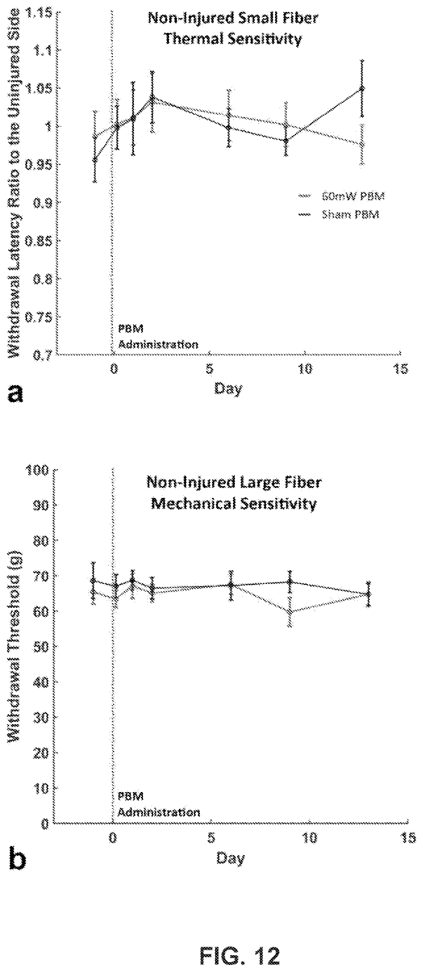

When PBM was delivered to sham SNI surgery animals (group 7), applying 60 mW for 4 minutes showed no reduction in heat hyperalgesia as compared to the sham SNI sham PBM group (group 1) over the observation time frame (see FIG. 12, elements a and b). As with the groups with SNI surgeries (groups 2-6), the mechanical hypersensitivity (von Frey response) was not affected with PBM application in healthy animals.

### Tissue Heating

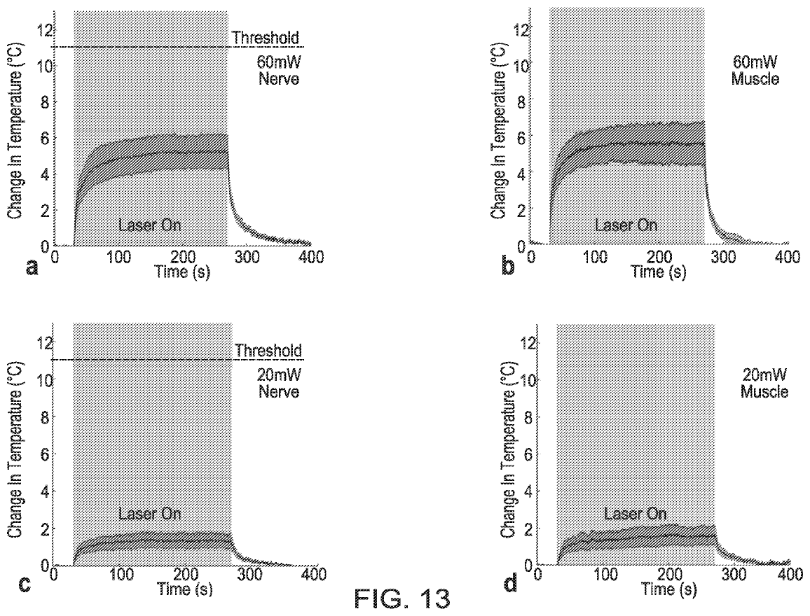

During PBM with an emitter power of 60 mW and duration of 4 minutes, the temperature of the nerve increased over time by approximately 5° C. (see FIG. 13, element a), and the time constant of the temperature rise was approximately 15 seconds. When applying 20 mW, the temperature increase at the nerve was approximately 1.33° C. (see FIG. 13, element c), and the time constant of the temperature rise was approximately 9 seconds. Note that in the case of 20 mW, the temperature reached a steady state by 4 minutes and dosing for 12 minutes did not raise the temperature further (data not shown). The time constant for the temperature return to baseline was approximately 11 seconds for both the 20 mW and 60 mW emitter powers. Applying these doses to the adjacent muscle resulted in increases in temperature of 5.6° C. and 1.6° C. for 60 mW (FIG. 13, element b) and 20 mW (FIG. 13, element d) emitter powers, respectively.

From the above description, those skilled in the art will perceive improvements, changes, and modifications. Such improvements, changes and modifications are within the skill of one in the art and are intended to be covered by the appended claims. All patents, patent applications, and publications cited herein are incorporated by reference in their entirety.

What is claimed is:

1. A method comprising:
configuring, by an external controller, a dose of PBM having at least one parameter; and
applying, by an emitter implanted proximal to at least one sensory and/or sensorimotor nerve of a patient and in communication with the controller for at most a time, the dose of PBM directly to the at least one sensory and/or sensorimotor nerve for the time to selectively inhibit conduction in at least one small diameter sensory nerve fiber in the at least one sensory and/or sensorimotor nerve without affecting conduction of at least one larger diameter nerve fiber in the at least one sensory and/or sensorimotor nerve, wherein conduction of the at least one sensory signal is inhibited for another time, wherein the other time is at least a day longer than the time.

2. The method of claim 1, wherein the at least one sensory signal is related to nociception.

3. The method of claim 1, wherein the at least one sensory signal is related to chronic pain of the patient.

4. The method of claim 1, wherein the other time is at least a week longer than the time.

5. The method of claim 1, wherein the other time is at least a month longer than the time.

6. The method of claim 1, wherein the emitter is implanted at most 3 mm from the at least one nerve without a barrier of natural tissue between the emitter and the at least one nerve.

7. The method of claim 1, wherein the applying the dose of PBM to the at least one sensorimotor nerve allows at least one mechanical signal to conduct through the at least one larger diameter nerve fiber in the at least one sensorimotor nerve normally.

8. The method of claim 1, wherein the parameter is related to a power density delivered to the nerve by the emitter, wherein the power density is delivered at a lower power than a power required for transcutaneous PBM to inhibit the at least one sensory signal.

9. The method of claim 1, wherein the configuring the dose of PBM further comprises retrieving the at least one parameter from a preset list of parameters based on a characteristic of the patient.

10. The method of claim 1, wherein the time comprises one hour or less and the other time comprises at least five days.

11. The method of claim 1, wherein the time comprises one hour or less and the other time comprises 30 days or more.

12. A system comprising:
an implantable emitter configured to be implanted in proximity to at least one sensory and/or sensorimotor nerve of a patient to deliver at least one dose of photobiomodulation (PBM) directly to the at least one sensory and/or sensorimotor nerve at an emitter power to inhibit conduction in at least one small diameter nerve fiber within the at least one sensory and/or sensorimotor nerve without affecting conduction of at least one larger diameter nerve fiber within the at least one sensory and/or sensorimotor nerve;
an external energy source located external to the emitter and temporarily coupled to the emitter for at most a time to drive delivery of the at least one dose of PBM from the emitter directly to the at least one sensory and/or sensorimotor nerve;
an external controller coupled to the emitter through the energy source for at most the time and configured to set at least one parameter for the at least one dose of PBM,
wherein application of the at least one dose of PBM for the time selectively inhibits at least a portion of conduction of at least one sensory signal by the at least one small diameter nerve fiber for another time, wherein the other time is at least a day longer than the time.

13. The system of claim 12, wherein the at least one parameter comprises at least one of a power, a wavelength, a duration, a dosing scheme, and a duty cycle.

14. The system of claim 12, wherein the at least one parameter comprises a dosing scheme that comprises a continuous application and/or a pulsatile application.

15. The system of claim 12, wherein at least one small diameter nerve fiber comprises at least one C-fiber.

16. The system of claim 12, wherein the time comprises one hour or less and the other time comprises at least five days.

17. The system of claim 12, wherein the time comprises one hour or less and the other time comprises 30 days or more.

18. The system of claim 12, wherein the dose of PBM applied to the at least one sensorimotor nerve allows at least one mechanical signal to conduct through the at least one larger diameter nerve fiber in the at least one sensorimotor nerve normally.
